SEQUENCE LISTING (34 sequences)

SEQ ID NO: 1 - DNA, 20 bp, Artificial sequence
  DNA Primer
gacacccgcattcaacaaac

SEQ ID NO: 2 - DNA, 20 bp, Artificial sequence
  DNA Primer
gagaagcaccttgacctgga

SEQ ID NO: 3 - DNA, 20 bp, Artificial sequence
  DNA Primer
cgccagcatgtctgataaac

SEQ ID NO: 4 - DNA, 19 bp, Artificial sequence
  DNA Primer
ctggctccttcatcggact

SEQ ID NO: 5 - DNA, 20 bp, Artificial sequence
  DNA Primer
agctgccccttatggaagat

SEQ ID NO: 6 - DNA, 20 bp, Artificial sequence
  DNA Primer
cctactgcttctcagcccac

SEQ ID NO: 7 - DNA, 20 bp, Artificial sequence
  DNA Primer
gtgtgcaggtcagaagcaga

SEQ ID NO: 8 - DNA, 20 bp, Artificial sequence
  DNA Primer
agcagtacggagctctgagg

SEQ ID NO: 9 - DNA, 20 bp, Artificial sequence
  DNA Primer
tcaggtgggagttcgtctca

SEQ ID NO: 10 - DNA, 21 bp, Artificial sequence
  DNA Primer
gcaggaatactgccatcaagg

SEQ ID NO: 11 - DNA, 20 bp, Artificial sequence
  DNA Primer
tgaccgcttacagaatgcag

SEQ ID NO: 12 - DNA, 19 bp, Artificial sequence
  DNA Primer
gagaaaaacgggccgcaag

SEQ ID NO: 13 - DNA, 20 bp, Artificial sequence
  DNA Primer
ggtggttggagagtccactg

SEQ ID NO: 14 - DNA, 20 bp, Artificial sequence
  DNA Primer
cttcatgaacatcctgggct

SEQ ID NO: 15 - DNA, 20 bp, Artificial sequence
  DNA Primer
agccccatcaagatgttcaa

SEQ ID NO: 16 - DNA, 18 bp, Artificial sequence
  DNA Primer
ggaaagggcaggatttcg

SEQ ID NO: 17 - DNA, 20 bp, Artificial sequence
  DNA Primer
aagatttgggcgcctctgtc

SEQ ID NO: 18 - DNA, 20 bp, Artificial sequence
  DNA Primer
atgcctctcttcttgccaaa

SEQ ID NO: 19 - DNA, 20 bp, Artificial sequence
  DNA Primer
tcaataccgactctggggag

SEQ ID NO: 20 - DNA, 20 bp, Artificial sequence
  DNA Primer
cctggcatctcattctcagc

SEQ ID NO: 21 - DNA, 20 bp, Artificial sequence
  DNA Primer
ccaggctttcgtgtttgtct

SEQ ID NO: 22 - DNA, 21 bp, Artificial sequence
  DNA Primer
gccaagatagaggtgaaacca

SEQ ID NO: 23 - DNA, 20 bp, Artificial sequence
  DNA Primer
catgtagccaagggaccagg

SEQ ID NO: 24 - DNA, 20 bp, Artificial sequence
  DNA Primer
cagagccatgaagacccgtt

SEQ ID NO: 25 - DNA, 20 bp, Artificial sequence
  DNA Primer
agggatgggctccaagttta

SEQ ID NO: 26 - DNA, 20 bp, Artificial sequence
  DNA Primer
ctcgcactttgtctgcatcg

SEQ ID NO: 27 - DNA, 20 bp, Artificial sequence
  DNA Primer
tggaggtgaggtctcgtgtt

SEQ ID NO: 28 - DNA, 20 bp, Artificial sequence
  DNA Primer
tggctaaacttcggttcaca

SEQ ID NO: 29 - DNA, 20 bp, Artificial sequence
  DNA Primer
ttggcagcgcctcctttaat

SEQ ID NO: 30 - DNA, 20 bp, Artificial sequence
  DNA Primer
tccaggatgtcaccaatgcc

SEQ ID NO: 31 - DNA, 20 bp, Artificial sequence
  DNA Primer
aacgtttctgaggaggggat

SEQ ID NO: 32 - DNA, 20 bp, Artificial sequence
  DNA Primer
tggtcaacacaatctgctcc

SEQ ID NO: 33 - DNA, 20 bp, Artificial sequence
  DNA Primer
ttcagtgtgttgcagaagcc

SEQ ID NO: 34 - DNA, 20 bp, Artificial sequence
  DNA Primer
cactgccacttgaggtcgat

(19) United States
(12) Patent Application Publication
(10) Pub. No.: US 2018/0078587 A1
(43) Pub. Date: Mar. 22, 2018

(54) COMPOSITIONS AND METHODS FOR PREVENTING COLORECTAL CANCER

(71) Applicant: TRUSTEES OF TUFTS COLLEGE, MEDFORD, MA (US)

(72) Inventor: Jimmy W. Crott, Medford, MA (US)

(21) Appl. No.: 15/558,835

(22) PCT Filed: Mar. 17, 2016
(86) PCT No.: PCT/US2016/022765

§ 371 Date: Sep. 15, 2017

Related U.S. Application Data
(60) Provisional application No. 62/134,922, filed on Mar. 18, 2015.

(51) Int. Cl. A61K 35/741; A61K 31/7076; A61K 9/50

(52) U.S. Cl. CPC A61K 35/741; A61K 9/50; A61K 31/7076

(57) ABSTRACT

Provided herein are compositions and methods for preventing and/or reducing the risk of colorectal cancer. In particular, provided herein are probiotic and small molecule agents and their use in preventing colorectal cancer.

The present Application claims priority to U.S. Provisional Patent Application Ser. No. 62/134,922 filed Mar. 18, 2015, the disclosure of which is herein incorporated by reference in its entirety.

### FIELD OF THE INVENTION

Provided herein are compositions and methods for preventing and/or reducing the risk of colorectal cancer. In particular, provided herein are probiotic and small molecule agents and their use in preventing colorectal cancer.

### BACKGROUND OF THE INVENTION

Colorectal cancer generally is a cancer from uncontrolled cell growth in the colon or rectum (parts of the large intestine) or in the appendix. Genetic analyses shows that essentially colon and rectal tumors are genetically the same cancer (see, e.g., Cancer Genome Atlas Network (19 Jul. 2012) Nature 487 (7407)). Symptoms of colorectal cancer typically include rectal bleeding and anemia which are sometimes associated with weight loss and changes in bowel habits.

Diagnosis of colorectal cancer is via tumor biopsy typically done during colonoscopy or sigmoidoscopy, depending on the location of the lesion. The extent of the disease is then usually determined by a CT scan of the chest, abdomen and pelvis. There are other potential imaging test such as PET and MRI which may be used in certain cases. Colon cancer staging is done next and based on the TMN system which is determined by how much the initial tumor has spread, if and where lymph nodes are involved, and if and how many metastases there are (see, e.g., Cunningham D, et al. (2010) Lancet 375 (9719): 1030-47).

At least 50% of the Western population will develop a colorectal tumor by age 70 years. In 10% of these individuals, the tumor progresses to malignancy. In adults, colorectal cancer is the second leading cancer that causes death worldwide (see, e.g., Bi X, et al., (2006) Mol Cell Proteomics 5(6):1119-30).

As such, improved techniques for detecting and preventing colorectal cancer are needed.

### SUMMARY OF THE INVENTION

Provided herein are compositions and methods for preventing and/or reducing the risk of colorectal cancer. In particular, provided herein are probiotic and small molecule agents and their use in preventing colorectal cancer.

For example, in some embodiments, the present disclosure provides a method of preventing colorectal cancer, comprising: providing a composition comprising adenosine and/or a composition comprising a bacterium of the species Parabacteroides to a subject. In some embodiments, the bacterium is Parabacteroides distasonis. In some embodiments, the subject is at risk for colorectal cancer (e.g., as result of a clinical finding selected from, for example, one or more of a family history of colorectal cancer, has previously had colorectal cancer, a finding of a polyp and/or precancerous lesion during colonoscopy or other diagnostic test, or a finding of a molecular marker associated with colorectal cancer). In some embodiments, the subject has been diagnosed with inflammatory bowel disease. In some embodiments, the subject has not been diagnosed with inflammatory bowel disease. In some embodiments, the subject is overweigh or obese. In some embodiments, the subject is not overweight or obese. In some embodiments, the bacterium and the adenosine are separately microencapsulated. In some embodiments, the bacterium and the adenosine are provided in a single composition.

Additional embodiments provide a composition comprising adenosine and a bacterium of the species Parabacteroides. In some embodiments, the composition is a pharmaceutical composition.

Further embodiments are described herein.

### BRIEF DESCRIPTION OF THE DRAWINGS

FIG. 1 shows impact of diet and genotype on body weight and tumor burden. A. Weight of female mice by group. B. Weight of male mice by group. C. Small intestinal tumor burden by group. ptrend <0.001 for tumor number and burden. Groups with different number are significantly different by post-test (p<0.05).

FIG. 2 shows LDA effect size analysis of between group differences in stool bacterial abundances in Apc1638N mice. A. Output showing effect size of all 29 significantly discriminant taxa. B. Taxa plotted onto a cladogram.

FIG. 3 shows the impact of obesity and tumor presence on the fecal metabolome of mice. First column (A-D), comparison of low and high fat fed mice; second column (E-H), comparison of low fat fed and genetically obese mice; third column (G-L) comparison of mice with and without tumors. Top row, heat map of significantly different metabolites (p<0.05); second row, volcano plots of significantly different metabolites (p<0.05); third row, discrimination of groups using Partial least squares discriminate analysis; fourth row, metabolites most strongly influencing discrimination by the partial lease squares discriminate analysis.

FIG. 4 shows an association of fecal adenosine concentration and Parabacteroides distasonis abundance with inflammatory cytokine production by the colonic mucosa. Normalized adenosine concentration in fecal matter correlates with Il1b and Tnf (B) but not Il4 (C) and Il6 (D) production in ex vivo colonic tissue. Relative abundance of Parabacteroides distasonis in fecal matter correlates with Il1b but not Tnf (B), Il4 (C) and Il6 (D) production in ex vivo colonic tissue.

FIG. 5 shows a heatmap of microbiome-metabolome interactions.

FIG. 6 shows A) LDA effect size (Lefse) output showing effect of group on microbiome. B) # of differently abundant operational taxonomic units for each comparison (p<0.05). C) Multivariate ‘Maaslin’ output showing negative association between P. distasonis & tumor number.

FIG. 7 shows A) No. of differentially abundant metabolites for each comparison (p<0.05). Adenosine concentrations for B) Apc LF v. Apc HF and Apc LF v. Apc DbDb and for C) tumor No v. Yes.

### DEFINITIONS

Following long-standing patent law convention, the terms “a”, “an”, and “the” refer to “one or more” when used in this application, including the claims. Thus, for example, reference to “a cell” or “a sample” includes a plurality of such cells or samples, respectively, and so forth.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “subject” as used herein includes all members of the animal kingdom including mammals, and suitably refers to humans. Optionally, the term “subject” includes mammals that have been diagnosed with a colorectal cancer or are in remission.

The term “biomolecule” refers to a molecule that is produced by a cell or tissue in an organism. Such molecules include, but are not limited to, molecules comprising nucleic acids, nucleotides, oligonucleotides, polynucleotides, amino acids, peptides, polypeptides, proteins, monoclonal and/or polyclonal antibodies, antigens, sugars, carbohydrates, fatty acids, lipids, steroids, and combinations thereof (e.g., glycoproteins, ribonucleoproteins, lipoproteins). Furthermore, the terms “nucleotide”, “oligonucleotide” or polynucleotide” refer to DNA or RNA of genomic or synthetic origin which may be single-stranded or double-stranded and may represent the sense or the antisense strand. Included as part of the definition of “oligonucleotide” or “polynucleotide” are peptide polynucleotide sequences (e.g., peptide nucleic acids; PNAs), or any DNA-like or RNA-like material (e.g., morpholinos, ribozymes).

The term “molecular entity” refers to any defined inorganic or organic molecule that is either naturally occurring or is produced synthetically. Such molecules include, but are not limited to, biomolecules as described above, simple and complex molecules, acids and alkalis, alcohols, aldehydes, arenas, amides, amines, esters, ethers, ketones, metals, salts, and derivatives of any of the aforementioned molecules.

The term “fragment” refers to a portion of a polynucleotide or polypeptide sequence that comprises at least a series (e.g., about 10, 15, 20, 30, etc.) consecutive nucleotides or 5 consecutive amino acid residues, respectively.

The terms “biological sample” and “test sample” refer to all biological fluids and excretions isolated from any given subject (e.g., a human patient diagnosed with colorectal cancer). In the context of the invention such samples include, but are not limited to, blood, serum, plasma, urine, semen, seminal fluid, seminal plasma, pre-ejaculatory fluid (Cowper's fluid), nipple aspirate, vaginal fluid, excreta, tears, saliva, sweat, biopsy, ascites, cerebrospinal fluid, lymph, marrow, hair or tissue extract samples.

The term “colorectal cancer” refers to a malignant neoplasm of the large intestine/colon within a given subject, wherein the neoplasm is of epithelial origin and is also referred to as a carcinoma of the large intestine/colon. According to the invention, colorectal cancer is defined according to its type, stage and/or grade. Typical staging systems known to those skilled in the art such as the Gleason Score (a measure of tumor aggressiveness based on pathological examination of tissue biopsy), the Jewett-Whitmore system and the TNM system (the system adopted by the American Joint Committee on Cancer and the International Union Against Cancer). The term “colorectal cancer”, when used without qualification, includes both localized and metastasised colorectal cancer. The term “colorectal cancer” can be qualified by the terms “localized” or “metastasised” to differentiate between different types of tumor as those words are defined herein. The terms “colorectal cancer” and “malignant disease of the large intestine/colon” are used interchangeably herein. The term “colorectal cancer” includes, but is not limited to, colon cancer, rectal cancer, and bowel cancer.

The terms “neoplasm” or “tumor” may be used interchangeably and refer to an abnormal mass of tissue wherein growth of the mass surpasses and is not coordinated with the growth of normal tissue. A neoplasm or tumor may be defined as “benign” or “malignant” depending on the following characteristics: degree of cellular differentiation including morphology and functionality, rate of growth, local invasion and metastasis. A “benign” neoplasm is generally well differentiated, has characteristically slower growth than a malignant neoplasm and remains localized to the site of origin. In addition a benign neoplasm does not have the capacity to infiltrate, invade or metastasize to distant sites. A “malignant” neoplasm is generally poorly differentiated (anaplasia), has characteristically rapid growth accompanied by progressive infiltration, invasion and destruction of the surrounding tissue. Furthermore, a malignant neoplasm has to capacity to metastasize to distant sites.

The term “metastasis” refers to the spread or migration of cancerous cells from a primary (original) tumor to another organ or tissue, and is typically identifiable by the presence of a “secondary tumor” or “secondary cell mass” of the tissue type of the primary (original) tumor and not of that of the organ or tissue in which the secondary (metastatic) tumor is located. For example, a colorectal cancer that has migrated to bone is said to be metastasised colorectal cancer, and consists of cancerous colorectal cancer cells in the large intestine/colon as well as cancerous colorectal cancer cells growing in bone tissue.

The term “differentially present” refers to differences in the quantity of a biomolecule present in samples taken from colorectal cancer patients or patients as increased risk of colorectal cancer as compared to samples taken from subjects having a non-malignant disease of the large intestine/colon or healthy subjects. Furthermore, a biomolecule is differentially present between two samples if the quantity of said biomolecule in one sample population is significantly different (defined statistically) from the quantity of said biomolecule in another sample population. For example, a given biomolecule may be present at elevated, decreased, or absent levels in samples of taken from subjects having colorectal cancer compared to those taken from subjects who do not have a colorectal cancer.

The term “diagnostic assay” can be used interchangeably with “diagnostic method” and refers to the detection of the presence or nature of a pathologic condition.

### DETAILED DESCRIPTION OF THE INVENTION

Provided herein are compositions and methods for preventing and/or reducing the risk of colorectal cancer. In particular, provided herein are probiotic and small molecule agents and their use in preventing colorectal cancer.

Provided herein are compositions and methods for preventing colorectal cancer. In some embodiments, compositions and methods utilize a bacterium of the genus Parabacteroides (e.g., Parabacteroides distasonis) and/or adenosine. In some embodiments, the bacterium and the adenosine are provided in the same or different compositions. In some embodiments, the adenosine and the bacterium are provided together in a single capsule, extract, pill, food product, supplement, or the like. In some embodiments, the bacterium and the adenosine are separately microencapsulated.

In some embodiments, the bacterium and the adenosine compositions are provide in a food or food product (e.g., a beverage, a yogurt, and the like). In some embodiments, the bacterium and the adenosine compositions are provided as a nutritional supplement (e.g., to be administered alone or added to a food or food product).

In some embodiments, the compositions described herein are administered with one or more additional agents (e.g. vitamin B6 and/or an anti-inflammatory agent (e.g., NSAID and/or other bacteria, especially species of the genus Lactobacillus).

In some embodiments, compositions comprising a bacterium and/or adenosine are administered to a subject at risk of colorectal cancer or a subject not at risk of colorectal cancer. In some embodiments, a subjects risk of colorectal cancer is determine by one or more of a family history of colorectal cancer, a finding of a polyp or precancerous lesion during colonoscopy, or a finding of a molecular marker associated with colorectal cancer (See e.g., Alquist, GASTROENTEROLOGY 2009; 136:2068-2073; herein incorporated by reference in its entirety), or prior diagnosis of colorectal cancer. In some embodiments, the subject has been diagnosed with inflammatory bowel disease. In some embodiments, the subject has not been diagnosed with inflammatory bowel disease. In some embodiments, the subject is overweight or obese. In some embodiments, the subject is not overweight or obese. In some embodiments the subject is at risk for colorectal cancer and is diagnosed with inflammatory bowel disease and is obese. In some embodiments, the subject is at risk of colorectal cancer and is not obese and has not been diagnosed with inflammatory bowel disease.

In some embodiments, the compositions are administered alone, while in some other embodiments, the compositions are preferably present in a pharmaceutical formulation comprising at least one active ingredient/agent, as defined above, together with a solid support or alternatively, together with one or more pharmaceutically acceptable carriers and optionally other therapeutic agents. Each carrier must be “acceptable” in the sense that it is compatible with the other ingredients of the formulation and not injurious to the subject.

Contemplated formulations include those suitable oral, rectal, nasal, topical (including transdermal, buccal and sublingual), vaginal, parenteral (including subcutaneous, intramuscular, intravenous and intradermal) and pulmonary administration. In some embodiments, formulations are conveniently presented in unit dosage form and are prepared by any method known in the art of pharmacy. Such methods include the step of bringing into association the active ingredient with the carrier which constitutes one or more accessory ingredients. In general, the formulations are prepared by uniformly and intimately bringing into association (e.g., mixing) the active ingredient with liquid carriers or finely divided solid carriers or both, and then if necessary shaping the product.

Formulations of the present invention suitable for oral administration may be presented as discrete units such as capsules, cachets or tablets, wherein each preferably contains a predetermined amount of the active ingredient; as a powder or granules; as a solution or suspension in an aqueous or non-aqueous liquid; or as an oil-in-water liquid emulsion or a water-in-oil liquid emulsion. In other embodiments, the active ingredient is presented as a bolus, electuary, or paste, etc.

Preferred unit dosage formulations are those containing a daily dose or unit, daily subdose, as herein above-recited, or an appropriate fraction thereof, of an agent.

It should be understood that in addition to the ingredients particularly mentioned above, the formulations of this invention may include other agents conventional in the art having regard to the type of formulation in question, for example, those suitable for oral administration may include such further agents as sweeteners, thickeners and flavoring agents. It also is intended that the agents, compositions and methods of this invention be combined with other suitable compositions and therapies. Still other formulations optionally include food additives (suitable sweeteners, flavorings, colorings, etc.), phytonutrients (e.g., flax seed oil), minerals (e.g., Ca, Fe, K, etc.), vitamins, and other acceptable compositions (e.g., conjugated linoelic acid), extenders, and stabilizers, etc.

Various delivery systems are known and can be used to administer compositions described herein, e.g., encapsulation in liposomes, microparticles, microcapsules, receptor-mediated endocytosis, and the like. Methods of delivery include, but are not limited to, intra-arterial, intra-muscular, intravenous, intranasal, and oral routes. In specific embodiments, it may be desirable to administer the pharmaceutical compositions of the invention locally to the area in need of treatment; this may be achieved by, for example, and not by way of limitation, local infusion during surgery, injection, or by means of a catheter.

Therapeutic amounts are empirically determined and vary with the pathology being treated, the subject being treated and the efficacy and toxicity of the agent. When delivered to an animal, the method is useful to further confirm efficacy of the agent.

In some embodiments, in vivo administration is effected in one dose, continuously or intermittently throughout the course of treatment. Methods of determining the most effective means and dosage of administration are well known to those of skill in the art and vary with the composition used for therapy, the purpose of therapy, the target cell being treated, and the subject being treated. Single or multiple administrations are carried out with the dose level and pattern being selected by the treating physician.

### EXPERIMENTAL

The following examples are provided in order to demonstrate and further illustrate certain preferred embodiments and aspects of the present invention and are not to be construed as limiting the scope thereof.

### Example 1

### Methods

### Animal Study

All animal procedures were approved by the institutional review board of the Jean Mayer USDA Human Nutrition Research Center on Aging at Tufts University. Three strains of mice were used for this study; wildtype C57BL6/J (Charles River, Wilmington, Mass.); Apc^{1638N} (NCI Mouse Repository. Frederick, Md.) and Lepr^{db} (Jackson Laboratory. Bar Harbor, Me.). Mice were individually housed on a 12 hr light-dark cycle at 23° C. and provided ad libitum access to water. To facilitate the study of intestinal tumorigenesis, the tumor-prone Apc^{1638N} mouse model was utilized. This mouse has a modification of exon 15 of one allele of the Apc gene, resulting in a chain-terminating truncation mutation of the Apc protein at codon 1638 (Fodde, R., W. Edelmann, K. Yang, C. van Leeuwen, C. Carlson, B. Renault, C. Breukel, E. Alt, M. Lipkin, P. M. Khan, and et al., A targeted chain-termination mutation in the mouse Apc gene results in multiple intestinal tumors. Proc Natl Acad Sci USA, 1994. 91(19): p. 8969-73). Mice heterozygous for this mutation spontaneously develop between 1-5 small bowel adenomas or carcinomas by the age of 8 months. In order to study genetically-induced obesity Lepr^{db/db} mice, which lack a functional Leptin rector and consequently become obese at 3-4 weeks of age, were used (Hummel, K. P., M. M. Dickie, and D. L. Coleman, Diabetes, a new mutation in the mouse. Science, 1966. 153(3740): p. 1127-8).

These mice were bred to generate the following three genotypes: Apc^{+/+}, Lepr^{+/+} (wildtype), Apc^{+/1638N}, Lepr^{+/+} (Apc) and Apc^{+/1638N}, Lepr^{db/db} (Apc-DbDb). Starting at 8 weeks of age, wildtype (n=12) and Apc-DbDb (n=10) mice were fed a low fat diet while Apc mice were randomized to receive low (N=10) or high (N=12) fat diet for 16 weeks. Low and high fat diets provided 10 and 60% of calories from fat respectively (Table 1. BioServ, Frenchtown, N.J.).

Mice were weighed weekly and after 15 weeks on diet body composition was measured by MRI (EchoMRI, Houston, Tex.). After 16 weeks on diet, mice were euthanized by CO₂ asphyxiation followed by cervical dislocation and exsanguination by cardiac puncture. The abdomen was then opened and the small intestine (SI) and large intestines removed onto separate ice-cold glass plates. Intestines were opened longitudinally and contents removed. Colon and cecum contents were combined, aliquoted, frozen in liquid N₂ and then stored at −80° C. Small and large intestines were then rinsed thoroughly with ice-cold PBS, then PBS with protease inhibitors (Roche, Indianapolis, Ind.). The small intestine was inspected for the presence of tumors by a blinded investigator under a dissecting microscope. Tumors were photographed and location and size noted before being excised and fixed in formalin for later grading by a rodent pathologist. The remaining normal-appearing SI mucosa, as well as the colonic mucosa, were scraped with microscope slides and frozen in liquid N₂ and then stored separately at −80° C. Liver, mesenteric fat and gonadal fat depots were also excised, weighed and frozen in N₂ and stored at −80° C. Blood was spun at 1000 g and plasma stored at −80° C. Plasma insulin and glucose concentrations were measured by ELISA and enzymatic colorimetric assays respectively (Millipore, Billerica, Mass.).

To assess colonic inflammation, two 1 cm sections of the colon were cultured for 24 hr in Dulbecco's Modified Eagle's Medium (DMEM) media with protease inhibitors (Roche, Indianapolis, Ind.) at 37° C. with 5% CO₂. After 24 hr, supernatant was collected and Il1b, Tnf, Il6 and Il4 were measured by electrochemiluminescence array and Sector S600 imager according to manufacturer's protocols (Mesoscale Discovery, Rockville, Md.).

### Fecal Metabolomics

Fecal samples (100 mg) were sent for non-targeted metabolic profiling (Metabolon, Durham, N.C.) as previously described (Ohta, T., N. Masutomi, N. Tsutsui, T. Sakairi, M. Mitchell, M. V. Milburn, J. A. Ryals, K. D. Beebe, and L. Guo, Untargeted metabolomic profiling as an evaluative tool of fenofibrate-induced toxicology in Fischer 344 male rats. Toxicol Pathol, 2009. 37(4): p. 521-35; Evans, A. M., C. D. DeHaven, T. Barrett, M. Mitchell, and E. Milgram, Integrated, nontargeted ultrahigh performance liquid chromatography/electrospray ionization tandem mass spectrometry platform for the identification and relative quantification of the small-molecule complement of biological systems. Anal Chem, 2009. 81(16): p. 6656-67). Briefly, lyophilized samples were analyzed by three independent platforms; ultrahigh performance liquid chromatography/tandem mass spectrometry (UHPLC/MS/MS) optimized for basic species, UHPLC/MS/MS optimized for acidic species, and gas chromatography/mass spectrometry (GC/MS). Metabolites were identified by automated comparison of the ion features in the experimental samples to a reference library of chemical standard entries that included retention time, molecular weight (m/z), preferred adducts, and in-source fragments as well as associated MS spectra, and were curated by visual inspection for quality control using software developed at Metabolon (Dehaven, C. D., A. M. Evans, H. Dai, and K. A. Lawton, Organization of GC/MS and LC/MS metabolomics data into chemical libraries. J Cheminform, 2010. 2(1): p. 9). For statistical analyses and data display purposes, any missing values were assumed to be below the limit of detection and these values were imputed with the compound minimum (minimum value imputation). Following median scaling and imputation of missing values, statistical analysis of (log-transformed) data was performed.

Metabolomic data were analyzed with MetaboAnalyst 2.0) (Xia, J., R. Mandal, I. V. Sinelnikov, D. Broadhurst, and D. S. Wishart, MetaboAnalyst 2.0—a comprehensive server for metabolomic data analysis. Nucleic Acids Res, 2012. 40(Web Server issue): p. W127-33). Data was normalized by sum and autoscaled. Heatmap visualization was performed based on Student's t-test results and reorganization of metabolites to show contrast between the groups. Red and blue colors in the heatmap indicate increased and decreased levels, respectively. Correction for multiple testing was done by calculating false discovery rate (FDR). Principal component analysis (PCA) and partial least-squares discriminant analysis (PLS-DA) were used for classification analyses. The Variable Importance In Projection (VIP) score is the weighted sum of squares for the partial least-squares loadings with the amount of y variance explained by each component taken into account. VIP score is given for each metabolite.

### Fecal Microbiome

DNA was extracted from frozen fecal samples using QiaAMP DNA Stool MiniKits (Qiagen, Valencia, Calif.) with modifications. The V4 region of the 16S rRNA gene was amplified using 12-base error-correcting Golay barcoded primers and PCR parameters as previously described (Caporaso, J. G., C. L. Lauber, W. A. Walters, D. Berg-Lyons, C. A. Lozupone, P. J. Turnbaugh, N. Fierer, and R. Knight, Global patterns of 16S rRNA diversity at a depth of millions of sequences per sample. Proc Natl Acad Sci USA, 2011. 108 Suppl 1: p. 4516-22). PCR reactions were carried out in triplicate in parallel with a barcode-specific negative control; reactions yielding no amplicon or those in which the negative controls amplified, were repeated. The amplicon pool was purified twice using an AMPure XP kit (Agencourt, Indianapolis, Ind.). Paired-end sequencing (250 bp) was performed on an Illumina HiSeq according to the manufacturer's protocols (SanDiego, Calif.). Computational analyses were performed using the open source software platform Qiime v 1.8.0 (Caporaso, J. G., J. Kuczynski, J. Stombaugh, K. Bittinger, F. D. Bushman, E. K. Costello, N. Fierer, A. G. Pena, J. K. Goodrich, J. I. Gordon, G. A. Huttley, S. T. Kelley, D. Knights, J. E. Koenig, R. E. Ley, C. A. Lozupone, D. McDonald, B. D. Muegge, M. Pirrung, J. Reeder, J. R. Sevinsky, P. J. Turnbaugh, W. A. Walters, J. Widmann, T. Yatsunenko, J. Zaneveld, and R. Knight, QIIME allows analysis of high-throughput community sequencing data. Nat Methods, 2010. 7(5): p. 335-6). After quality filtering using Qiime default parameters, paired-end sequences were concatenated and demultiplexed. Closed reference OTUs at 99% similarity were assigned using Greengenes (DeSantis, T. Z., P. Hugenholtz, N. Larsen, M. Rojas, E. L. Brodie, K. Keller, T. Huber, D. Dalevi, P. Hu, and G. L. Andersen, Greengenes, a chimera-checked 16S rRNA gene database and workbench compatible with ARB. Appl Environ Microbiol, 2006. 72(7): p. 5069-72) and an OTU table was generated. The classification data was used to generate comparisons of relative abundance of selected phyla or genera between samples. The number of sequences were normalized to 41000 (minimum read depth returned) and phylotype-based alpha diversity measures including equitability, number of observed species, Shannon diversity index, Chao-1 and phylogenetic distance were determined. Differences in OTU abundance according to group and other traits were identified using the LDA Effect Size (Lefse) and Multivariate Association with Linear Models (MaAsLin) tools of Huttenhower (Segata, N., J. Izard, L. Waldron, D. Gevers, L. Miropolsky, W. S. Garrett, and C. Huttenhower, Metagenomic biomarker discovery and explanation. Genome Biol, 2011. 12(6): p. R60).

### Gene Expression

The expression of several adenosine-metabolizing genes in the small intestinal mucosa were profiled: adenosine deaminase (Ada) converts adenosine to inosine; adenosine kinase (Adk) forms AMP from adenosine and ATP; ectonucleoside triphosphate diphosphohydrolases (Entpd1/3/8) convert ATP to ADP and AMP; purine nucleoside phosphoylases (Pnp, Pnp2) metabolize adenosine into adenine; S-adenosylhomocysteine hydrolase (Ahcy) catalyzes the hydrolysis of S-adenosylhomocysteine to adenosine and L-homocysteine; deoxycytidine kinase (Dck) converts AMP to adenosine, 5′ nucleotidases convert AMP to adenosine (nt5 c/c1a/c1b/c2/c3/c3b/e/m). Total RNA was isolated from small intestinal scrapings using Trizol reagent and cDNA synthesized using Superscript III reverse transcriptase. Real-time PCR was performed using SYBR green master mix (Life technologies, Grand Island, N.Y.) and an ABI7300 thermocyler (Applied Biosystems, Foster City, Calif.). Primer sequences for each gene of interest were obtained from qPrimerDepot or NCBI Primer Blast (Ye, J., G. Coulouris, I. Zaretskaya, I. Cutcutache, S. Rozen, and T. L. Madden, Primer-BLAST: a tool to design target-specific primers for polymerase chain reaction. BMC Bioinformatics, 2012. 13: p. 134) and are listed in Table 4. Relative expression was calculated using the 2^{-ΔΔCt} method and statistical analyses were performed on ΔCt values. Gapdh was used as the control gene.

### Statistics

All data is reported as mean±SEM. Statistical calculations were performed in Systat (San Jose, Calif.) and R. Between groups comparisons were made with ANOVA, 2way ANOVA or T-test were appropriate. Associations between variables were assessed by linear regression. Significance was accepted when p<0.05 and, when multiple comparisons conducted, a False Discovery Rate with a cutoff of q<0.2 was used. Cluster analysis and heatmaps were generated with CIMminer.

### Results

### Physiology

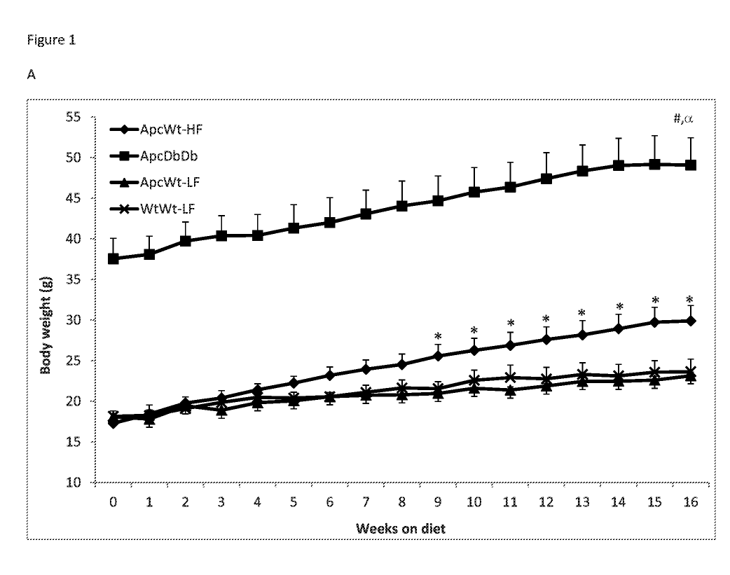

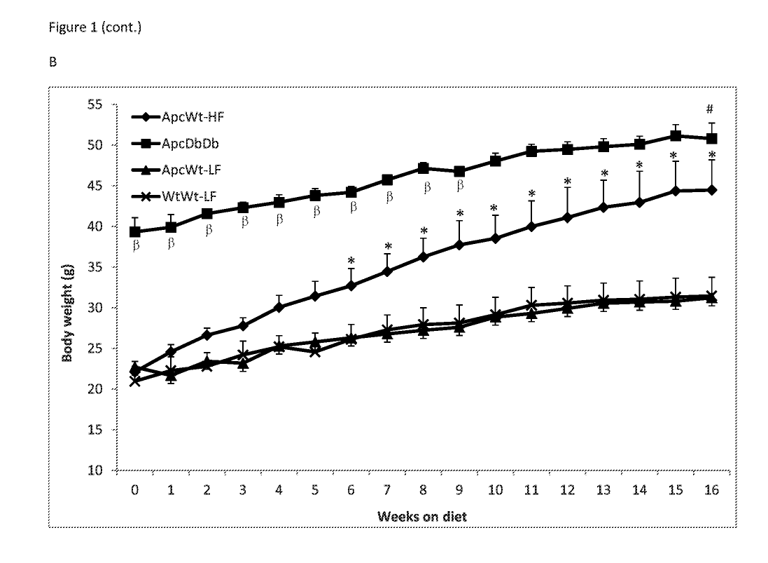

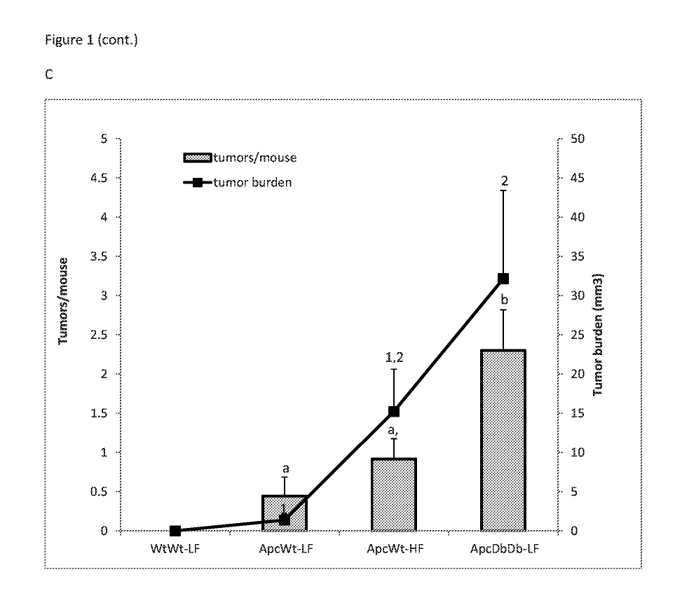

High fat consumption increased body weight in both male and female mice, an effect that attained statistical significance after 9 wk in females and 6 wk in males. Apc-DbDb mice began the diet period approximately double the body weight of all other mice. Amongst females, Apc-DbDb's remained significantly heavier than all other groups for the duration of the intervention but for males the difference between Apc-DbDb and Apc-HF mice disappeared after 10 wk on diet (FIG. 1A,B). At wk 15 body composition was determined by MRI; fat mass was significantly higher in both male and female Apc-DbDb mice and although numerically higher in female HF mice, only attained statistical significance in male HF. Lean mass was not altered by HF consumption or DbDb genotype in either sex. Liver weight was greatly elevated in DbDb mice of both sexes. Insulin and glucose were not significantly elevated in the HF group, but were elevated substantially in DbDb mice (Table 2).

### Intestinal Tumors

No tumors were observed in the WT-LF mice. Amongst Apc^{1638N} mice the tumor incidence was 33%, 67% and 100% in LF, HF and DbDb mice respectively (χp<0.005). A similar significant step-wise increase in tumor multiplicity and burden was also observed (FIG. 1C). All tumors were histologically confirmed to be adenomatous polyps.

### Fecal Microbiome

Population diversity was assessed by via several metrics. Significant between-group differences were observed with Observed Species and PD whole tree metrics (p<0.05), while a trend was apparent for Chao index (p=0.064). For these analyses the Apc-HF group had the lowest numerical value which attained significance in comparison with the Apc-DbDb group. No significant differences were observed between groups for Shannon index or Equitability index (p>0.05). When comparing between groups at a phylum level there were no significant differences in the four major phyla present (Actinobacteria, Bacteroidetes, Firmicutes, Proteobacteria) or in the ratio of Firmicutes:Bacteroidetes (ANOVA p>0.05).

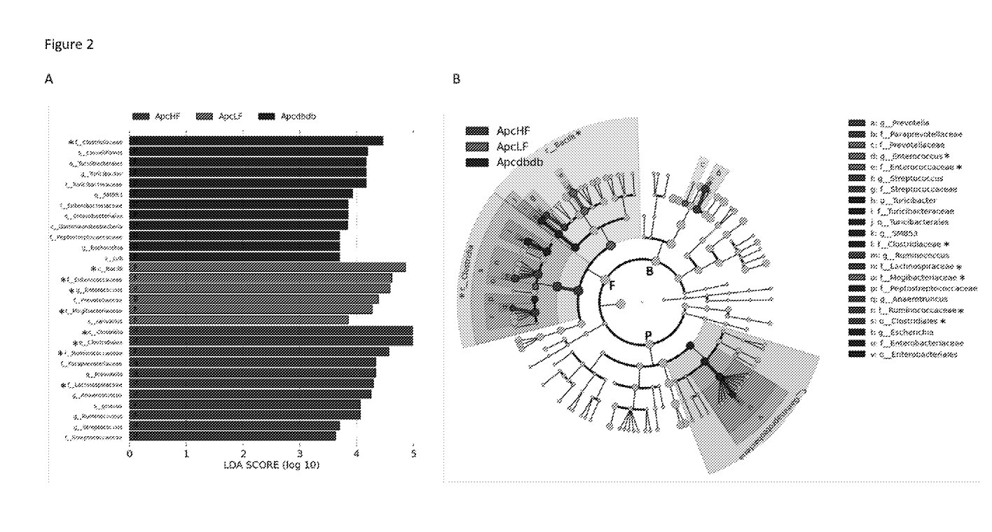

LDA effect size analyses was performed on data from Apc^{1638N} mice and identified 29 significantly enriched features across three phyla; 6, 11 and 12 taxa for LF, HF and DbDb mice respectively (FIG. 2A,B). Taxa belonging to the phylum Firmicutes featured prominently amongst those enriched in both modes of obesity (9 of 11 and 6 of 12 for HF and DbDb respectively). For HF mice the remainder of these defining taxa were of the phylum Bacteroidetes (2 of 11) while for DbDb an equal number belong to the phylum Proteobacteria (6 of 12). Multivariate analysis of microbial community structure with MaAsLin facilitated parsing out associations with genotype, diet, sex and tumor number (Table 3). In agreement with the LEfSe analysis, the family Clostridiacea (phyla Firmicutes) was associated with the DbDb genotype; families Ruminococcaceae and Lachnospiracea (both phyla Firmicutes) were associated with high fat diet and the family Enterococcaceae (phyla Firmicutes) was associated with the low fat diet. In addition several OTUs from Firmicutes and Bacteroidetes were associated with each sex.

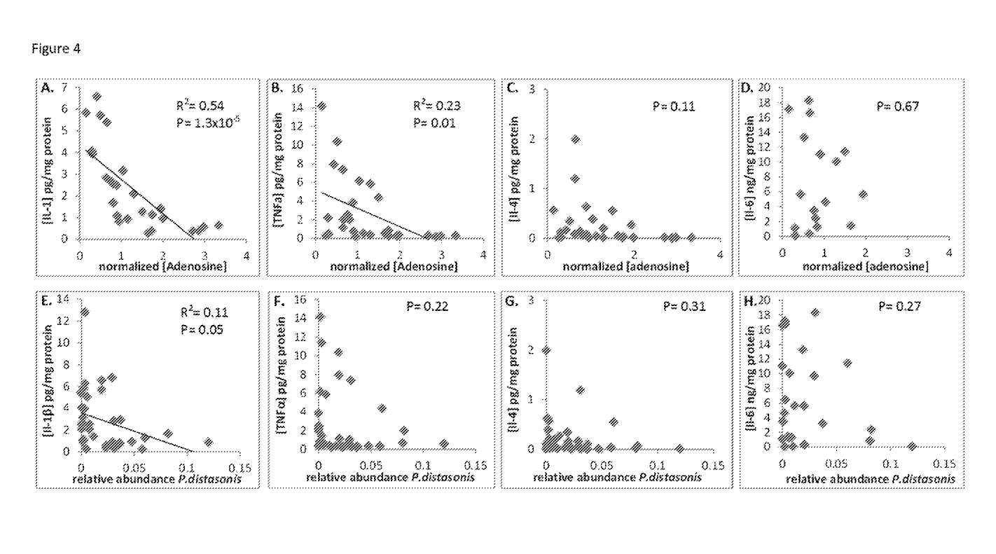

MaAsLin also identified OTUs both positively (phyla Firmicutes and Actinobacteria) and negatively (phyla Bacteroidetes) associated with tumor number. Amongst these, Parabacteroides distasonis was also identified by Lefse analysis as being depleted in tumor-bearing mice. Further, simple t-test (p=0.02) and regression analyses (R=−0.31, p=0.04) confirmed a depletion of P. distasonis in tumor-bearing mice and with increasing tumor number respectively. The relative abundance of P. distasonis was also inversely related to colonic production of Il1b (R=−0.34, p=0.05) but not Tnf, Il6 or Il4 (P>0.05) (FIG. 4).

### Fecal Metabolome

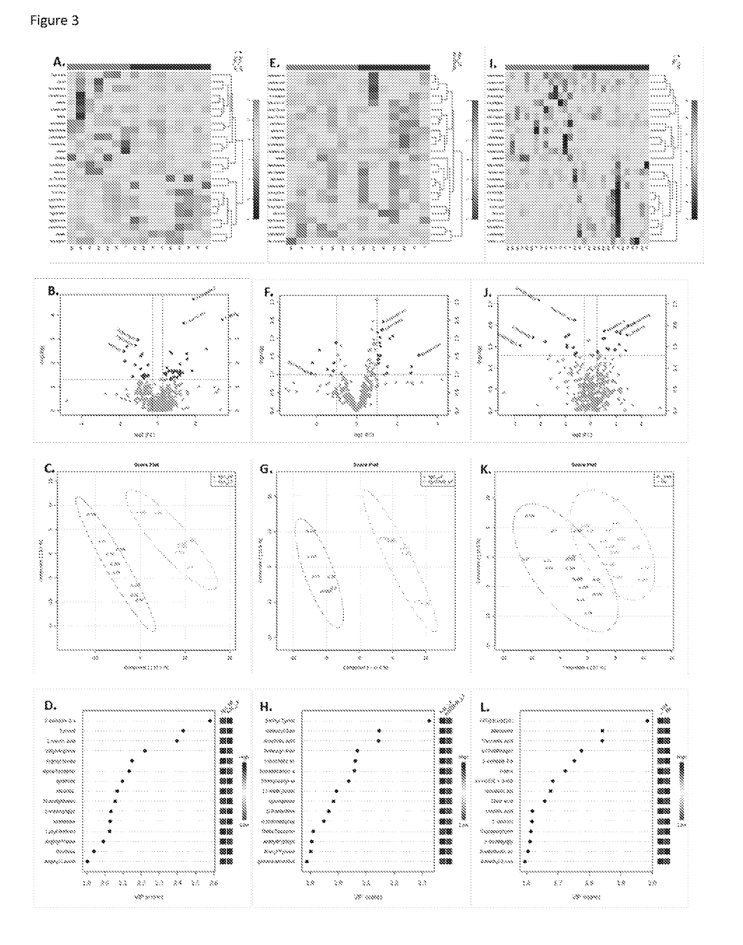

415 metabolites were detected in the sample set. Data were normalized and between-group comparisons made with t-tests in MetaboAnalyst. Comparing Apc-LF and Apc-HF mice, 49 metabolites returned a p value of <0.05 and 14 with a q<0.2 (FIG. 3A,B). Comparing Apc-LF and Apc-DbDb mice 41 metabolites returned a p value of <0.05 but 0 attained a q<0.2 (FIG. 3E,F). Using the relaxed cut-off of p<0.05, 5 metabolites were changed in both comparisons: adenosine, 2-oxindole-3-acetate, caproic acid, arachadic acid and tyrosyl glycine. Comparing mice with and without tumors, 29 metabolites returned a p value of <0.05 but 0 attained a q<0.2 (FIG. 3I,J). Adenosine and 2-oxindole-3-acetate were altered in all three comparisons.

Because previous studies clearly indicate an anti-inflammatory role for adenosine in the colon, its association with inflammatory cytokine production in the colon was tested. Consistent with such a role, fecal adenosine concentrations were significantly inversely associated with the abundance of pro-inflammatory cytokines Tnf (R=−0.5,p=0.01) and Il1b (R=−0.73, p=1.3×10⁻⁵) but not Il4 or Il6 (p>0.05) (FIG. 4).

Adenosine may enter 3 metabolic pathways which begin with the formation of AMP, adenine and inosine. To investigate possible mechanisms for the observed depletion of adenosine, its association with these proximal metabolites and also the genes responsible for these reactions was tested. Fecal adenosine was positively associated with inosine (R=0.167, p=0.03) but not adenine. AMP was not detected in the sample set. Of the AMP-forming genes, adenosine concentration was significantly inversely related to the expression of Adk (R²=0.37, p=0.001) but not Dck, Entpd1 (CD39), Entpd3, Entpd8, nt5c, nt5c1a, nt5c1b, nt5c2, nt5c3, nt5c3b, nt5e (CD73) or nt5m (p>0.05). Adenosine concentration was unrelated to the expression of adenine-forming genes Pnp and Pnp2 or the inosine-forming gene Ada (p>0.05).

Using the relaxed cut-off Partial Least Squares Discriminate Analysis could effectively separate Apc-HF and Apc-DbDb groups from the Apc-LF group (FIG. 3C,G). The metabolites that most heavily drove this discrimination are 2-oxindole-3-acetate, tyrosol and Lactic acid for the HF comparison and serinyl tyrosine, isoleucyl serine and arachidic acid for the DbDb comparison (FIG. 3D,H). Similarly mice with and without tumors could be distinguished in this analysis, with oleic acid, adenosine and vaccenic acid being most influential (FIG. 3K,L). In contrast, Principle Component Analyses could not effectively distinguish groups in these two comparisons.

### Integrative Analysis

Correlation analysis between all OTUs and metabolites revealed that 107 metabolites and 31 OTUs had at least one significant association (q<0.05). a cluster analysis of the correlation R values was performed and 2 clear clusters of bacteria were observed, indicating similarities in their metabolic capacities and/or requirements (FIG. 5). Cluster 1 is comprised mostly of members of the class bacilli while Cluster 2 is made up of 3 classes of proteobacteria (beta, delta, gamma), class clostridia and class TM7-3 of phyla TM7.

While the concentration of adenosine was not significantly associated with the abundance of any OTU (q>0.2), its immediate precursor adenine was strongly associated with the genus Lactobacillus (R=0.75, q=0.002) and 3 other higher order taxa associated with this genus (family lactobacillaceae, order lactobacillales and class bacilli. R=0.75-0.65, q=0.002-0.03).

TABLE 1

| | LFD | HFD |
|---|---|---|
| Ingredient (g/kg) | | |
| Casein | 210 | 265 |
| L-Cystine | 3 | 4 |
| Corn Starch | 280 | 0 |
| Maltodextrin | 50 | 160 |
| Sucrose | 325 | 90 |
| Lard | 20 | 310 |
| Soybean Oil | 20 | 30 |
| Cellulose | 37.2 | 65.5 |
| Mineral Mix AlN-93G | 35 | 48 |
| Calcium Phosphate Dibasic | 2 | 3.4 |
| Vitamin Mix AlN-93 | 15 | 21 |
| Choline Bitartrate | 2.8 | 3 |
| Total | 1000 | 1000 |
| Energy (% kcal) | | |
| Carbohydrate | 70 | 21 |
| Protein | 20 | 19 |
| Fat | 10 | 60 |
| Total | 100 | 100 |

TABLE 1
Diet composition.
LFD, Low fat diet. HFD, High fat diet. BioServ catalogue numbers F6654, and F6653 respectively.

| | Wt Wt | | Apc LF | | Apc HF | |
|---|---|---|---|---|---|---|
| Endpoint | M (7) | F (5) | M (5) | F (5) | M (4) | F (8) |
| Body weight (g) | 31.31 ± 2.33 | 23.58 ± 1.42 | 30.83 ± 1.80 | 22.60 ± 0.46 | 44.41 ± 3.66* | 29.73 ± 1.83 |
| Total fat mass(g) | 8.05 ± 1.70 | 5.07 ± 0.89 | 7.60 ± 1.41 | 4.85 ± 1.04 | 19.13 ± 3.08* | 9.94 ± 1.96 |
| Total lean mass (g) | 18.95 ± 0.85 | 15.24 ± 0.86 | 18.66 ± 0.32 | 14.27 ± 1.14 | 21.06 ± 1.17 | 16.75 ± 0.37 |
| Mesenteric fat (g) | 0.55 ± 0.11 | 0.28 ± 0.07 | 0.41 ± 0.07 | 0.28 ± 0.04 | 1.23 ± 0.28* | 0.39 ± 0.09 |
| Gonadal fat (g) | 1.01 ± 0.24 | 0.58 ± 0.15 | 0.97 ± 0.18 | 0.52 ± 0.09 | 2.48 ± 0.26* | 1.53 ± 0.35 |
| Liver (g) | 1.29 ± 0.17 | 0.94 ± 0.10 | 1.19 ± 0.06 | 1.04 ± 0.07 | 1.33 ± 0.15 | 0.96 ± 0.04 |
| Plasma Insulin | 3.10 ± 1.26 | 0.94 ± 0.16 | 1.70 ± 0.23 | 1.20 ± 0.20 | 4.09 ± 1.91 | 1.09 ± 0.18 |
| (ng/ml) | | | | | | |
| Plasma Glucose | 8.09 ± 0.87 | 5.08 ± 2.56 | 8.21 ± 0.42 | 7.78 ± 0.47 | 11.19 ± 1.07 | 10.05 ± 0.92 |
| (μM) | | | | | | |

| | Apc DbDb | | 2Way ANOVA P | |
|---|---|---|---|---|
| Endpoint | M (3) | F (7) | Group | Sex |
| Body weight (g) | 51.13 ± 1.38* | 49.17 ± 3.51* | <0.0001 | <0.0001 |
| Total fat mass(g) | 28.22 ± 1.04* | 26.51 ± 1.93* | <0.0001 | 0.006 |
| Total lean mass (g) | 18.89 ± 0.62 | 17.39 ± 1.56 | 0.1 | <0.0001 |
| Mesenteric fat (g) | 1.04 ± 0.16 | 1.00 ± 0.13* | <0.0001 | 0.002 |
| Gonadal fat (g) | 1.57 ± 0.18 | 1.84 ± 0.26* | <0.0001 | 0.063 |
| Liver (g) | 4.93 ± 0.30* | 3.63 ± 0.29* | <0.0001 | <0.0001 |
| Plasma Insulin | 11.44 ± 0.82 | 16.03 ± 2.43* | <0.0001 | 0.8 |
| (ng/ml) | | | | |
| Plasma Glucose | 20.81 ± 1.73* | 18.26 ± 2.51* | <0.0001 | 0.1 |
| (μM) | | | | |

TABLE 2
Physiological characteristics of mice by group.

| Variable | Feature (OTU) | Coefficient | P-value | Q-value |
|---|---|---|---|---|
| Apc WT | p_Actinoc_Actinobacteria | 0.00 | 0.004 | 0.097 |
| Apc WT | p_Proteoc_Gammaproteoo_Pseudomonadales | 0.00 | 0.010 | 0.163 |
| Apc WT | p_Bacteroidetes \| c_Bacteroidia \| o_Bacteroidales \| f_Paraprevotellaceae | −0.08 | 0.012 | 0.163 |
| Apc WT | p_Bacteroidetes \| c_Bacteroidia \| o_Bacteroidales \| f_Paraprevotellaceae \| g_Prevotella | −0.08 | 0.012 | 0.163 |
| Apc WT | p_Actinoc_Actinoo_Bifidobacteriales \| f_Bifidobacteriaceae | 0.00 | 0.013 | 0.163 |
| Apc WT | p_Actinoc_Actinoo_Bifidobacteriales \| f_Bifidobacteriaceae \| g_Bifidobacterium | 0.00 | 0.013 | 0.163 |
| Apc WT | p_Firmicutes \| c_Clostridia \| o_Clostridiales \| f_Peptococcaceae | −0.02 | 0.018 | 0.197 |
| DbDb WT | p_Firmicutes \| c_Clostridia \| o_Clostridiales \| f_Clostridiaceae \| g_Sarcina | 0.00 | 0.002 | 0.055 |
| DbDb WT | p_Bacteroidetes \| c_Bacteroidia \| o_Bacteroidales \| f_Paraprevotellaceae | 0.09 | 0.007 | 0.128 |
| DbDb WT | p_Bacteroidetes \| c_Bacteroidia \| o_Bacteroidales \| f_Paraprevotellaceae \| g_Prevotella | 0.09 | 0.007 | 0.128 |
| DbDb WT | p_Bacteroidetes \| c_Bacteroidia \| o_Bacteroidales \| f_Rikenellaceae | 0.12 | 0.014 | 0.170 |
| DbDb WT | p_Bacteroidetes \| c_Bacteroidia \| o_Bacteroidales \| f_Prevotellaceae | 0.01 | 0.014 | 0.170 |
| DbDb WT | p_Firmicutes \| c_Clostridia \| o_Clostridiales \| f_Clostridiaceae | −0.10 | 0.017 | 0.192 |
| DbDb WT | p_Firmicutes \| c_Bacilli \| o_Lactobacillales \| f_Carnobacteriaceae | 0.00 | 0.018 | 0.197 |
| LF Diet | p_Firmicutes \| c_Clostridia \| o_Clostridiales \| f_Ruminococcaceae | −0.11 | 4.03E−05 | 0.024 |
| LF Diet | p_Firmicutes \| c_Clostridia \| o_Clostridiales \| f_Ruminococcaceae \| g_Anaerotruncus | −0.01 | 0.000 | 0.040 |
| LF Diet | p_Firmicutes \| c_Bacilli \| o_Lactobacillales \| f_Enterococcaceae \| g_Enterococcus | 0.17 | 0.000 | 0.040 |
| LF Diet | p_Firmicutes \| c_Bacilli \| o_Lactobacillales \| f_Enterococcaceae | 0.17 | 0.001 | 0.040 |
| LF Diet | p_Firmicutes \| c_Bacilli \| o_Lactobacillales \| f_Enterococcaceae \| | 0.01 | 0.001 | 0.040 |
| | g_Enterococcus \| s_casseliflavus | | | |
| LF Diet | p_Firmicutes \| c_Clostridia \| o_Clostridiales \| f_Lachnospiraceae \| g_Roseburia | −0.03 | 0.001 | 0.040 |
| LF Diet | p_Firmicutes \| c_Clostridia \| o_Clostridiales \| f_Peptostreptococcaceae | 0.03 | 0.001 | 0.040 |
| LF Diet | p_Firmicutes \| c_Clostridia \| o_Clostridiales \| f_Mogibacteriaceae | 0.01 | 0.002 | 0.062 |
| LF Diet | p_Bacteroidetes \| c_Bacteroidia \| o_Bacteroidales \| f_S24-7 | 0.11 | 0.005 | 0.114 |
| LF Diet | p_Firmicutes \| c_Bacilli \| o_Turicibacterales | 0.05 | 0.011 | 0.163 |
| LF Diet | p_Firmicutes \| c_Bacilli \| o_Turicibacterales \| f_Turicibacteraceae | 0.05 | 0.011 | 0.163 |
| LF Diet | p_Firmicutes \| c_Bacilli \| o_Turicibacterales \| f_Turicibacteraceae \| g_Turicibacter | 0.05 | 0.011 | 0.163 |
| LF Diet | p_Firmicutes \| c_Clostridia \| o_Clostridiales \| f_Clostridiaceae \| g_SMB53 | 0.06 | 0.012 | 0.163 |
| LF Diet | p_Firmicutes \| c_Clostridia \| o_Clostridiales \| f_Lachnospiraceae | −0.06 | 0.012 | 0.163 |
| LF Diet | p_Firmicutes \| c_Clostridia \| o_Clostridiales \| f_Lachnospiraceae \| g_Coprococcus | −0.02 | 0.012 | 0.163 |
| LF Diet | p_Firmicutes \| c_Bacilli | 0.17 | 0.014 | 0.170 |
| LF Diet | p_Firmicutes \| c_Clostridia | −0.15 | 0.017 | 0.192 |
| LF Diet | p_Firmicutes \| c_Clostridia \| o_Clostridiales | −0.15 | 0.017 | 0.192 |
| Male Sex | p_Firmicutes | 0.15 | 0.000 | 0.040 |
| Male Sex | p_Firmicutes \| c_Clostridia \| o_Clostridiales \| f_Lachnospiraceae \| g_Dorea | −0.01 | 0.001 | 0.040 |
| Male Sex | p_Bacteroidetes \| c_Bacteroidia \| o_Bacteroidales \| f_Porphyromonadaceae | −0.08 | 0.001 | 0.040 |
| Male Sex | p_Bacteroidetes \| c_Bacteroidia \| o_Bacteroidales \| | −0.08 | 0.001 | 0.040 |
| | f_Porphyromonadaceae \| g_Parabacteroides | | | |
| Male Sex | p_Bacteroidetes | −0.16 | 0.001 | 0.040 |
| Male Sex | p_Bacteroidetes \| c_Bacteroidia | −0.16 | 0.001 | 0.040 |
| Male Sex | p_Bacteroidetes \| c_Bacteroidia \| o_Bacteroidales | −0.16 | 0.001 | 0.040 |
| Male Sex | p_Bacteroidetes \| c_Bacteroidia \| o_Bacteroidales \| | −0.07 | 0.003 | 0.083 |
| | f_Porphyromonadaceae \| g_Parabacteroides \| s_distasonis | | | |
| Male Sex | p_Firmicutes \| c_Clostridia \| o_Clostridiales \| f_Dehalobacteriaceae | −0.01 | 0.008 | 0.150 |
| Male Sex | p_Firmicutes \| c_Clostridia \| o_Clostridiales \| f_Dehalobacteriaceae \| | −0.01 | 0.009 | 0.163 |
| | g_Dehalobacterium | | | |
| Male Sex | p_Firmicutes \| c_Clostridia \| o_Clostridiales \| f_Lachnospiraceae \| g_Coprococcus | −0.02 | 0.013 | 0.163 |
| Male Sex | p Firmicutes \| c Bacilli | 0.14 | 0.014 | 0.170 |
| Tumor # | p_Bacteroidetes \| c_Bacteroidia \| o_Bacteroidales \| f_Porphyromonadaceae | −0.05 | 0.001 | 0.040 |
| Tumor # | p_Bacteroidetes \| c_Bacteroidia \| o_Bacteroidales \| | −0.05 | 0.001 | 0.040 |
| | f_Porphyromonadaceae \| g_Parabacteroides | | | |
| Tumor # | p_Bacteroidetes \| c_Bacteroidia \| o_Bacteroidales \| | −0.04 | 0.001 | 0.051 |
| | f_Porphyromonadaceae \| g_Parabacteroides \| s_distasonis | | | |
| Tumor # | p_Actinobacteria \| c_Actinobacteria \| o_Actinomycetales \| f_Corynebacteriaceae | 0.02 | 0.004 | 0.093 |
| Tumor # | p_Actinobacteria \| c_Actinobacteria \| o_Actinomycetales \| | 0.02 | 0.004 | 0.093 |
| | f_Corynebacteriaceae \| g_Corynebacterium | | | |
| Tumor # | p_Bacteroidetes | −0.09 | 0.004 | 0.093 |
| Tumor # | p_Bacteroidetes \| c_Bacteroidia | −0.09 | 0.004 | 0.093 |
| Tumor # | p_Bacteroidetes \| c_Bacteroidia \| o_Bacteroidales | −0.09 | 0.004 | 0.093 |
| Tumor # | p_Actinobacteria \| c_Actinobacteria \| o_Actinomycetales \| | 0.01 | 0.004 | 0.097 |
| | f_Micrococcaceae \| g_Arthrobacter | | | |
| Tumor # | p_Actinobacteria \| c_Actinobacteria \| o_Actinomycetales | 0.02 | 0.005 | 0.105 |
| Tumor # | p_Firmicutes \| c_Bacilli \| o_Lactobacillales \| f_Aerococcaceae \| g_Aerococcus | 0.02 | 0.010 | 0.163 |
| Tumor # | p Firmicutes | 0.06 | 0.012 | 0.163 |

Total lean and fat mass measured by MRI.
M, male; F, female.
Samples size in parentheses.
* P < 0.05 vs Ape LF (of same sex).

TABLE 3
Multivariate Association with Linear Models (MaAsLin) output.
Model = Apc (Mut or Wt), DbDb (Mut or Wt), Diet (LF or HF),
Sex (M or F) and Tumors (number of tumors present). Mut, mutatnt; Wt, wildtype;
LF, low fat; HF, high fat. N = 41. Taxa in bold were also identified to be associated
with that trait (variable) in the LDA effect size analysis.

| | Gene | mRNA | SEQ ID | | | SEQ ID | Amplicon |
|---|---|---|---|---|---|---|---|
| Gene name | Symbol | Refseq# | NO: | Left primer | Right primer | NO: | length |
| adenosine | Ada | NM_007398.4 | 1 | GACACCCGCATTCAA | ATGCCTCTCTTCTTGC | 18 | 99 bp |
| deaminase | | | | CAAAC | CAAA | | |
| adenosine kinase | Adk | NM_134079.4 | 2 | GAGAAGCACCTTGAC | TCAATACCGACTCTGG | 19 | 103 bp |
| | | | | CTGGA | GGAG | | |
| S-adenosyl | Ahcy | NM_016661.3 | 3 | CGCCAGCATGTCTGA | CCTGGCATCTCATTCT | 20 | 99 bp |
| homocysteine | | | | TAAAC | CAGC | | |
| hydrolase | | | | | | | |
| deoxycytidine | Dck | NM_007832.4 | 4 | CTGGCTCCTTCATCGG | CCAGGCTTTCGTGTTT | 21 | 107 bp |
| kinase | | | | ACT | GTCT | | |
| ectonucleoside | Entpd1 | NM_009848.3 | 5 | AGCTGCCCCTTATGG | GCCAAGATAGAGGTG | 22 | 94 bp |
| triphosphate | (CD39) | | | AAGAT | AAACCA | | |
| diphos- | | | | | | | |
| phohydrolase 1 | | | | | | | |
| ectonucleoside | Entpd3 | NM_178676.4 | 6 | CCTACTGCTTCTCA | CATGTAGCCAAGGG | 23 | 134 bp |
| triphosphate | | | | GCCCAC | ACCAGG | | |
| diphos- | | | | | | | |
| phohydrolase 3 | | | | | | | |
| ectonucleoside | Entpd8 | NM_028093.1 | 7 | GTGTGCAGGTCAGA | CAGAGCCATGAAGA | 24 | 115 bp |
| triphosphate | | | | AGCAGA | CCCGTT | | |
| diphos- | | | | | | | |
| phohydrolase 8 | | | | | | | |
| 5′,3′- | Nt5c | NM_015807.1 | 8 | AGCAGTACGGAGCTC | AGGGATGGGCTCCAA | 25 | 92 bp |
| nucleotidase, | | | | TGAGG | GTTTA | | |
| cytosolic | | | | | | | |
| 5′-nucleotidase, | Nt5c1a | NM_001085502.1 | 9 | TCAGGTGGGAGTTC | CTCGCACTTTGTCT | 26 | 149 bp |
| cytosolic IA | | | | GTCTCA | GCATCG | | |
| 5′-nucleotidase, | Nt5c1b | NM_027588.3 | 10 | GCAGGAATACTGCC | TGGAGGTGAGGTCT | 27 | 95 bp |
| cytosolic IB | | | | ATCAAGG | CGTGTT | | |
| 5′-nucleotidase, | Nt5c2 | NM_029810.4 | 11 | TGACCGCTTACAGAA | TGGCTAAACTTCGGTT | 28 | 110 bp |
| cytosolic II | | | | TGCAG | CACA | | |
| 5′-nucleotidase, | Nt5c3 | NM_026004.3 | 12 | GAGAAAAACGGGCC | TTGGCAGCGCCTCC | 29 | 129 bp |
| cytosolic III | | | | GCAAG | TTTAAT | | |
| 5′-nucleotidase, | Nt5c3b | NM_001102650.1 | 13 | GGTGGTTGGAGAGT | TCCAGGATGTCACC | 30 | 117 bp |
| cytosolic IIIB | | | | CCACTG | AATGCC | | |
| 5′ nucleotidase, | Nt5e | NM_011851.4 | 14 | CTTCATGAACATCCTG | AACGTTTCTGAGGAG | 31 | 97 bp |
| ecto | (CD73) | | | GGCT | GGGAT | | |
| 5′,3′- | Nt5m | NM_134029.2 | 15 | AGCCCCATCAAGATG | TGGTCAACACAATCT | 32 | 97 bp |
| Nucleotidase, | | | | TTCAA | GCTCC | | |
| mitochondrial | | | | | | | |
| purine- | Pnp | NM_013632.4 | 16 | GGAAAGGGCAGGATT | TTCAGTGTGTTGCAG | 33 | 104 bp |
| nucleoside | | | | TCG | AAGCC | | |
| phosphorylase | | | | | | | |
| purine- | Pnp2 | NM_001123371.2 | 17 | AAGATTTGGGCGCC | CACTGCCACTTGAG | 34 | 116 bp |
| nucleoside | | | | TCTGTC | GTCGAT | | |
| phosphorylase 2 | | | | | | | |
| | Gapdh | | | | | | |

TABLE 4
Gene expression primers for murine adenosine-metabolizing genes.
Ada-did transcript variant 2 not 1
Nt5c2-transcript variant 3 not 1
** not in q primer. Min 80, max 150 crossing exon exon

### Example 2

Parabacteroides distasonis and Adenosine as Anti-Inflammatory Agents to Prevent Cancer.

Five to six percent of the US population will develop colorectal cancer (CRC) in their lifetime. This translates to 137,000 new cases and 50,000 deaths from CRC per year (Siegel, R., C. Desantis, and A. Jemal, Colorectal cancer statistics, 2014. CA Cancer J Clin, 2014. 64(2): p. 104-17). Among the many risk factors for CRC is obesity, a condition afflicting 36% of the US population. Obese individuals have a 50-100% increased risk of developing CRC compared to lean individuals (Calle, E. E. and R. Kaaks, Overweight, obesity and cancer: epidemiological evidence and proposed mechanisms. Nat Rev Cancer, 2004. 4(8): p. 579-91) and compelling evidence indicates that elevated inflammation constitutes a major mechanistic link (Aleman, J. O., L. H. Eusebi, L. Ricciardiello, K. Patidar, A. J. Sanyal, and P. R. Holt, Mechanisms of obesity-induced gastrointestinal neoplasia. Gastroenterology, 2014. 146(2): p. 357-73; Yehuda-Shnaidman, E. and B. Schwartz, Mechanisms linking obesity, inflammation and altered metabolism to colon carcinogenesis. Obes Rev, 2012. 13(12): p. 1083-95). Despite current efforts to control obesity, it is clear that a substantial percentage of the population will remain obese, and have higher rates of CRC, for the foreseeable future.

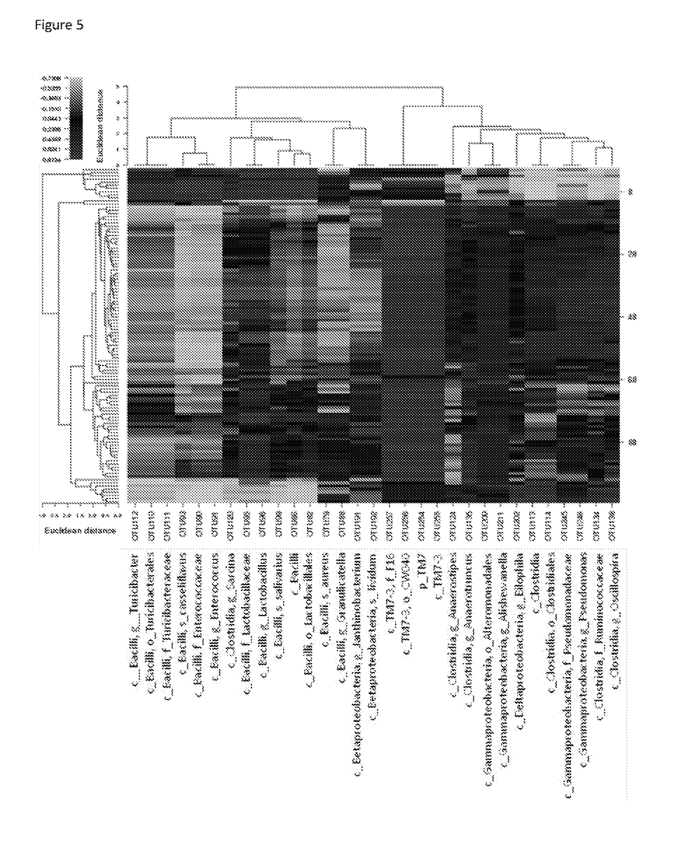

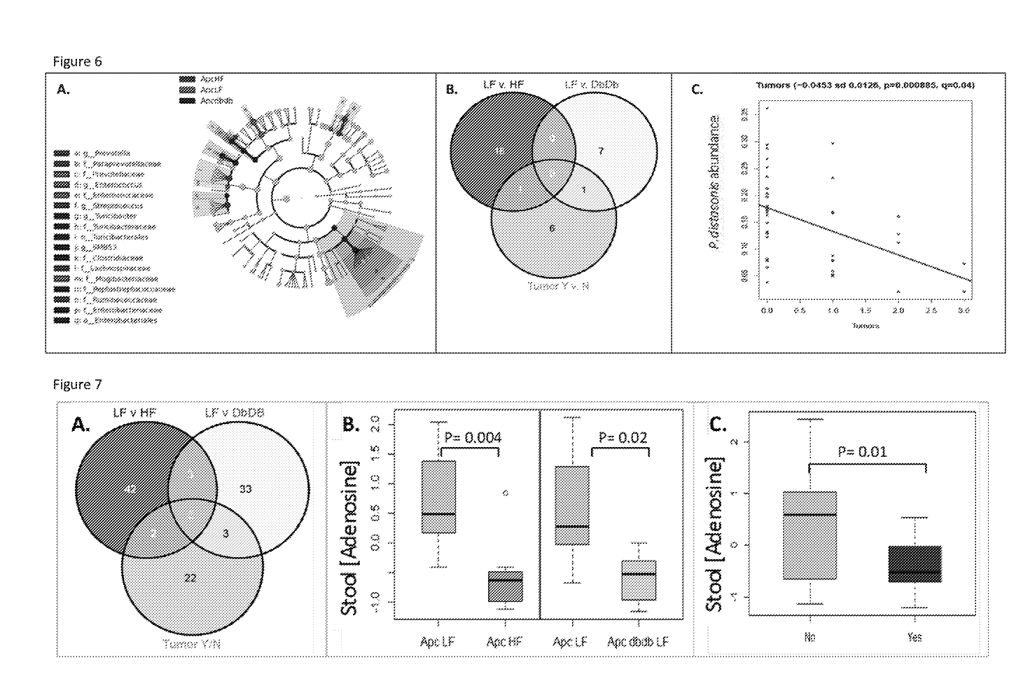

One likely avenue by which obesity might promote CRC is by causing a shift in the “demographics” of the gut bacterial population, or microbiome, to one that is more pro-inflammatory. The studies described herein were designed to further understanding in this regard. Apc^{1638N} mice, which spontaneously form intestinal tumors, were made obese by high fat (HF) feeding or with an obesogenic mutation (Lepr^{db/db}) and their gut microbiome was compared to low fat (LF) fed Apc^{1638N} mice. Many changes in the gut microbiome were observed with high fat feeding and relatively fewer with genetic obesity (FIG. 6A,B). Multivariate analyses (Maaslin) taking into account mouse genotype, gender and diet revealed an inverse association between the species Parabacteroides distasonis and tumor burden (FIG. 6C). Univariate models including LDA effect size as well as simple t-tests (p=0.02) and Pearson correlations (R=−0.33, p=0.03) corroborate this association.

In addition to characterizing the microbiome, untargeted metabolomics of the stool were performed to gain a deeper understanding how obesity impacts on the intestinal milieu. 415 metabolites were identified; 49 were altered by high fat consumption and 41 by genetic obesity (P<0.05). Comparing mice with and without tumors, there were 29 differentially abundant biochemicals (FIG. 7A). Only adenosine and 2-oxindole-3-acetate were altered in all three comparisons (FIG. 7A-C). Adenosine is of great interest because it is well documented to be anti-inflammatory in the colon. In the study stool adenosine was strongly negatively associated with mucosal abundance of pro-inflammatory cytokines Tnf (R=−0.5,p=0.01) and Il1b (R=−0.73, p=1.3×10⁻⁵).

Thus studies have identified two novel entities that are depleted in obesity and in the presence of tumors. Depletion of these entities promotes, or is permissive, in the development of obesity-associated colonic inflammation which results in a pro-tumorigenic milieu. Strategies to restore levels are employed to reduce the risk for CRC.

1. A method of preventing colorectal cancer, comprising:
providing a composition comprising adenosine and/or a composition comprising a bacterium of the species Parabacteroides to a subject.

2. The method of claim 1, wherein said bacterium is Parabacteroides distasonis.

3. The method of claim 1, wherein said subject is at risk for colorectal cancer.

4. The method of claim 3, wherein said risk is the result of a clinical finding selected from the group consisting of a family history of colorectal cancer, a prior history of colorectal cancer, a finding of a polyp or precancerous lesion during colonoscopy, and a finding of a molecular marker associated with colorectal cancer.

5. The method of claim 1, wherein said subject has been diagnosed with inflammatory bowel disease.

6. The method of claim 1, wherein said subject has not been diagnosed with inflammatory bowel disease.

7. The method of claim 1, wherein said subject is overweight or obese.

8. The method of claim 1, wherein said subject is not overweight or obese.

9. The method of claim 1, wherein said bacterium and said adenosine are separately microencapsulated.

10. The method of claim 1, wherein said bacterium and said adenosine are provided in a single composition.

11. A composition comprising adenosine and a bacterium of the genus Parabacteroides.

12. The composition of claim 11, wherein said bacterium is Parabacteroides distasonis.
